(19) United States
(12) Patent Application Publication
(10) Pub. No.: US 2008/0230073 A1
(43) Pub. Date: Sep. 25, 2008

(54) Vibrating prophylactic

(75) Inventor: Simon Siu Man Nan, Richmond Hill (CA)

Correspondence Address: GREENSFELDER HEMKER & GALE PC, SUITE 2000, 10 SOUTH BROADWAY, ST LOUIS, MO, 63102, US

(73) Assignee: Nanma Manufacturing Co. Ltd., Chai Wan (HK)

(21) Appl. No.: 11/724,930

(22) Filed: Mar. 16, 2007

(51) Int. Cl. A61F 6/04

(52) U.S. Cl. 128/844

(57) ABSTRACT

A prophylactic device, as one or more embodiments, includes a vibration generator and a switch disposed in a distal end of the device. The vibration generator is capable of being activated by pushing the switch towards a proximal end of the device. As one or more embodiments, a switching apparatus is provided for activating a vibration generator, which apparatus includes a ring including a recess, a switch including at least one arm connected to the ring, and an electrically conductive arm configured to contact a pole of a motor when the ring is in an activated position and to contact the recess when the ring is in a deactivated position.

### CROSS-REFERENCE TO RELATED APPLICATIONS

Not applicable.

### BACKGROUND

The presently described invention generally relates to prophylactics. More specifically, embodiments of the presently described technology provide an improved electrically powered vibrating condom.

In use, existing vibrating massagers used as sexual aid devices are commonly held by hand. Such use by a female prevents simultaneous intercourse with a male partner. Thus, only the female partner obtains a benefit from the existing vibrating massager.

Other existing devices include a miniature vibrating device worn on the base of a male partner's sexual organ for providing vibrating stimulation to a female partner's genitals during intercourse. However, this type of device is limited to stimulating the external portions of the female partner's sexual organs and can not stimulate the internal part of these organs. These internal portions of the female partner's organs can provide increased stimulation to the female partner.

It is desirable to provide male and female partners with a vibrating actuator during intercourse that does not have an adverse effect on either partner but enhances each partner's enjoyment. Thus, a need exists for a vibrating massager that can provide stimulation to both partners during intercourse and/or provide stimulation to an internal part of the female partner during intercourse with her male partner.

### BRIEF SUMMARY

One or more embodiments of the presently described invention provides a prophylactic device that includes a vibration generator and a switch disposed in a distal end of the device. The vibration generator is capable of being activated by pushing the switch towards a proximal end of the device.

One or more embodiments of the presently described invention also provides a method for providing a vibrating prophylactic device. The method includes providing a vibration generator and a switch in a distal end of the device and activating the vibration generator by pushing the switch towards a proximal end of the device.

One or more embodiments of the presently described invention also provides a switching apparatus for activating a vibration generator. The apparatus includes a ring including a recess, a switch including at least one arm connected to the ring, and an electrically conductive arm configured to contact a pole of a motor when the ring is in an activated position and configured to contact the recess when the ring is in a deactivated position.

### BRIEF DESCRIPTION OF THE DRAWINGS

FIG. 1 illustrates an orthogonal view of a vibrating condom device in accordance with an embodiment of the presently described invention.

FIG. 2 illustrates a cross-sectional view of the device in accordance with an embodiment of the presently described invention.

FIG. 3 illustrates an orthogonal view of the device before the vibration generator and switch are inserted into the vibration actuator section of the device in accordance with an embodiment of the presently described invention.

FIG. 4 illustrates a first perspective view of the vibration generator in accordance with an embodiment of the presently described invention.

FIG. 5 illustrates a second perspective view of the vibration generator in accordance with an embodiment of the presently described invention.

FIG. 6 illustrates a third perspective view of the vibration generator in accordance with an embodiment of the presently described invention.

FIG. 7 illustrates an exploded view of the vibration generator in accordance with an embodiment of the presently described invention.

FIG. 8 illustrates a flowchart for a method of providing a vibrating prophylactic in accordance with an embodiment of the presently described invention.

The foregoing summary, as well as the following detailed description of certain embodiments of the presently described technology, will be better understood when read in conjunction with the appended drawings. For the purpose of illustrating the presently described technology, certain embodiments are shown in the drawings. It should be understood, however, that the presently described technology is not limited to the arrangements and instrumentality shown in the attached drawings.

### DETAILED DESCRIPTION

Embodiments of the presently described invention relate to an electrically powered vibrating condom. For example, one or more embodiments include a condom having a miniature or micro-vibrating actuator inside a condom for providing stimulation to an internal part of a female partner during intercourse. In an embodiment, the actuator provides stimulation to the female partner's so-called “G-spot” during intercourse.

In a preferred embodiment, the inventive device described herein comprises a skin, a vibration actuator, a power source and a power switch. The device is preferred for a single time use. That is, the device is preferably used only a single time and then disposed. In another embodiment, however, the device can be designed for multiple uses.

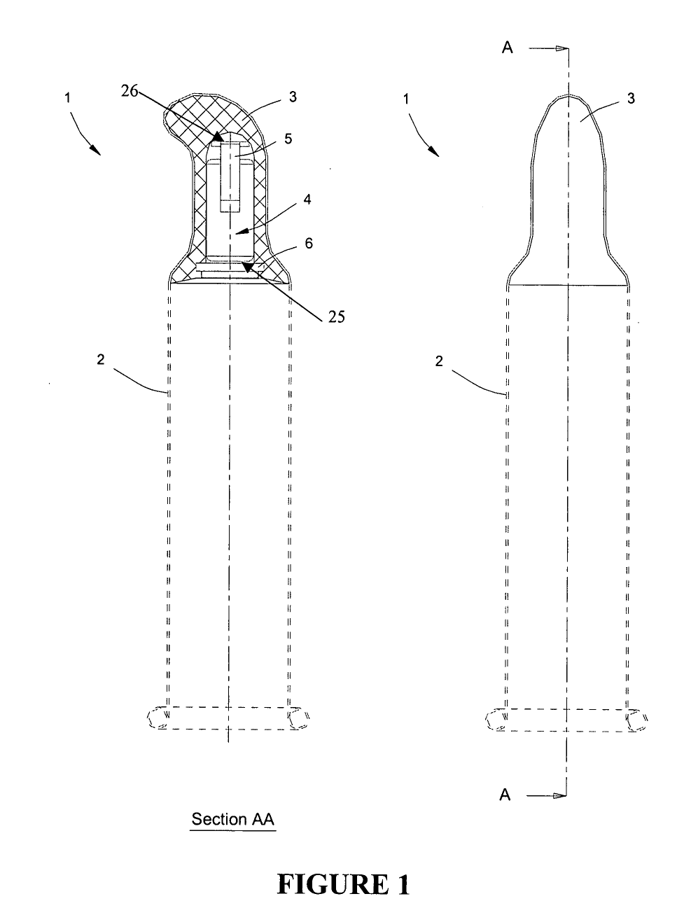

FIG. 1 illustrates an orthogonal view of a vibrating condom device 1 in accordance with an embodiment of the presently described invention. Device 1 includes a skin 2, a vibration actuator section 3, a vibration generator 4, a power switch 5 and a cover 6.

Vibrator actuator section 3 is disposed at a distal end of device 1. Section 3 can be curved along one or more dimensions. For example, as shown in FIG. 1, section 3 can be curved along one direction or dimension and not curved along any other direction or dimension. Such curvature can assist in providing stimulation to specific parts of a female anatomy. For example, by curving section 3 upwards (or in a transverse direction or a direction perpendicular with respect to the longitudinal axis of device 1) with respect to the male sex organ, device 1 and specifically section 3 can provide stimulation to a female partner's so-called “G-spot” during intercourse.

Skin 2 can comprise a resilient or elastic material. For example, skin 2 can comprise a traditional prophylactic or condom material. In an embodiment, section 3 comprises the same material as the remainder of device 1. For example, all of device 1, including section 3, can comprise skin 2. In another embodiment, section 3 comprises a material with a different resiliency or elasticity than the remainder of device 1. For example, the material in section 3 can be stiffer than the material of skin 2 for the remainder of device 1.

Vibration generator 4 and power switch 5 can be enclosed in section 3 by cover 6. That is, cover 6 can enclose vibration generator 4 and switch 5 within section 3 by preventing or inhibiting generator 4 and switch 5 from falling out of section 3, for example. Cover 6 can be made from the same or different material as skin 2. In an embodiment, cover 6 is made from a resilient or elastic material. In another embodiment, cover 6 is made from a stiff or inelastic material.

Power switch 5 can activate and/or deactivate vibration of vibration generator 4. For example, when power switch 5 is pulled away from generator 4, generator 4 is deactivated or is “OFF” and therefore does not vibrate. When switch 5 is pushed in towards generator 4, generator 4 is activated or is “ON” and therefore vibrates.

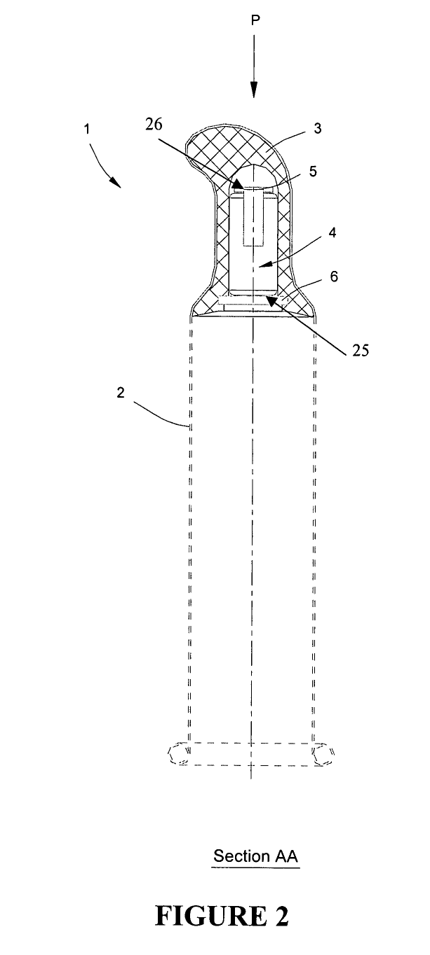

FIG. 2 illustrates a cross-sectional view of device 1 in accordance with an embodiment of the presently described invention. As shown in FIG. 2, power switch 5 is in a down, or activated position, thus causing generator 4 to vibrate and cause section 3 to vibrate.

In an embodiment, switch 5 is capable of being pushed down into the “ON” position (as shown in FIG. 2) and pulled up into the “OFF” position (as shown in FIG. 1) a plurality of times. That is, generator 4 can be turned ON and OFF more one time.

In another embodiment, prior to use of device 1, switch 5 is set in the OFF position shown in FIG. 1 and is capable of being pushed down into the ON position shown in FIG. 2 only once. That is, once generator 4 is activated by pushing down on switch 5, generator 4 cannot be turned off or deactivated until after device 1 has been used or the power source for generator 4 is depleted or removed.

In an embodiment, switch 5 can only be activated by pushing switch 5 towards a proximal end 25 of device 1, or downwards, as shown by direction P in FIG. 2. That is, generator 4 cannot be activated or turned on by pushing switch 5 towards a distal end 26 of device 1, or upwards or in a direction opposite P, on generator 4 or cover 6. In other words, in this embodiment, switch 5 can only be turned to the ON position by pushing it downwards towards generator 4. Such an inability can result from generator 4 being fixed in position with respect to section 3 with switch 5 being moveable (and thus not fixed with respect to section 3), for example.

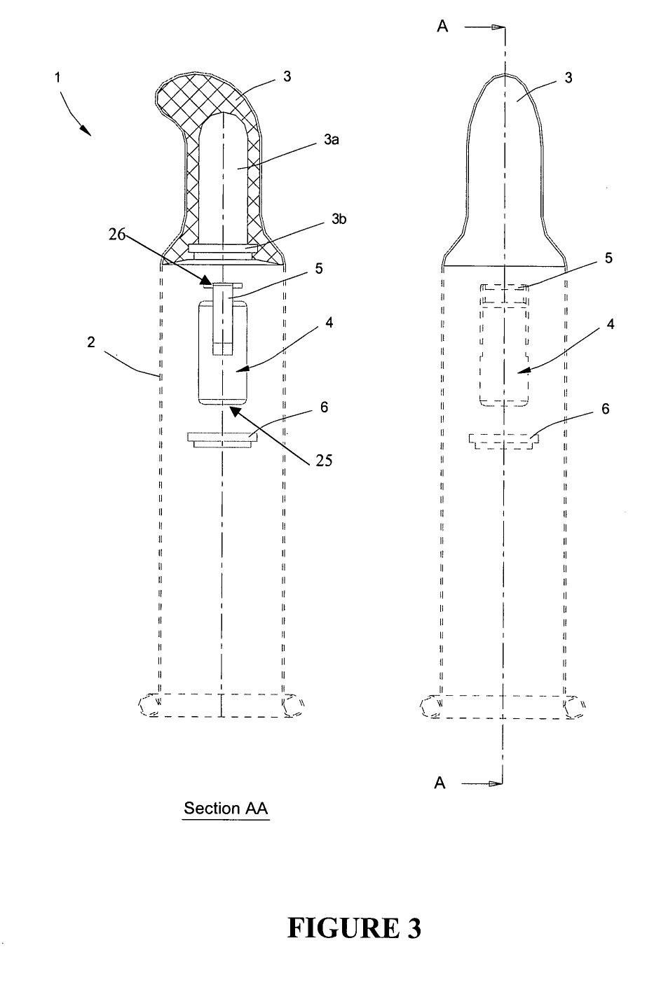

FIG. 3 illustrates an orthogonal view of device 1 before vibration generator 4 and switch 5 are inserted into vibration actuator section 3 in accordance with an embodiment of the presently described invention. Prior to assembly of device 1, switch 5 can be pulled away from generator 4 (thus placing vibration generator 4 in the deactivated or “OFF” position) and then inserted into an empty or hollow chamber 3a of section 3. Cover 6 can then be placed inside an annular groove 3b of section 3, thus enclosing generator 4 and switch 5 inside section 3.

In an embodiment, cover 6 is fixed in annular groove 3b. That is, cover 6 is attached to groove 3b in such a manner such that removal of cover 6 would cause damage to skin 2, section 3 and/or cover 6, for example. Cover 6 can be fixed in groove 3b by use of an adhesive or by melting or otherwise bonding cover 6 to skin 2 of section 3, for example.

In another embodiment, cover 6 is removable from annular groove 3b. That is, cover 6 is capable of being placed in groove 3b and removed from groove 3b a plurality of times without causing damage to skin 2, section 3 and/or cover 6, for example. In such an embodiment, vibration generator 4 and switch 5 can be removed from one device 1 after its use and inserted into another device 1.

In an embodiment, generator 4 is fixed or adhered to cover 6. For example, generator 4 can be adhesively secured, as by glue, or otherwise bonded to cover 6. In another embodiment, generator 4 is not fixed or adhered to cover 6.

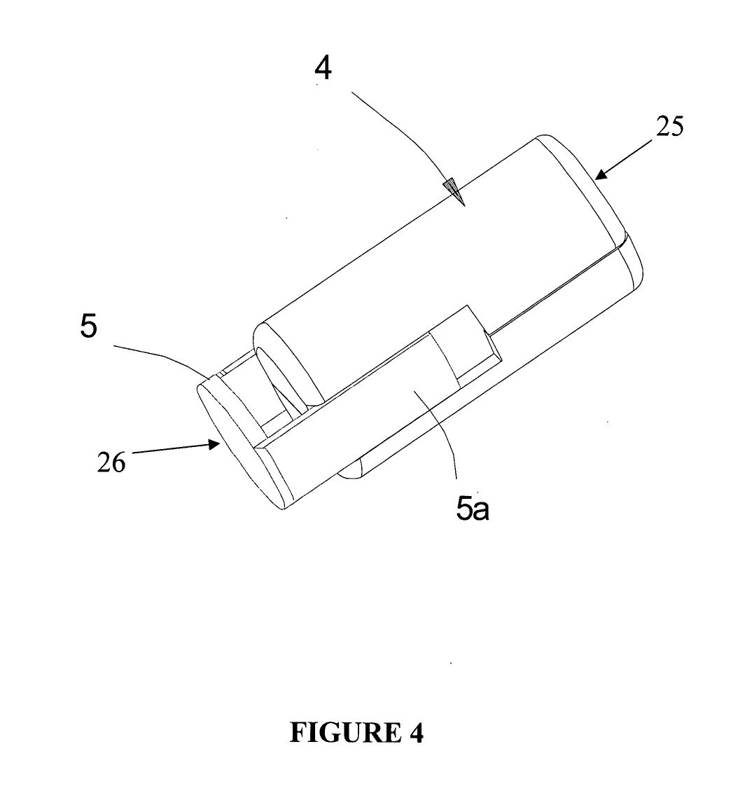

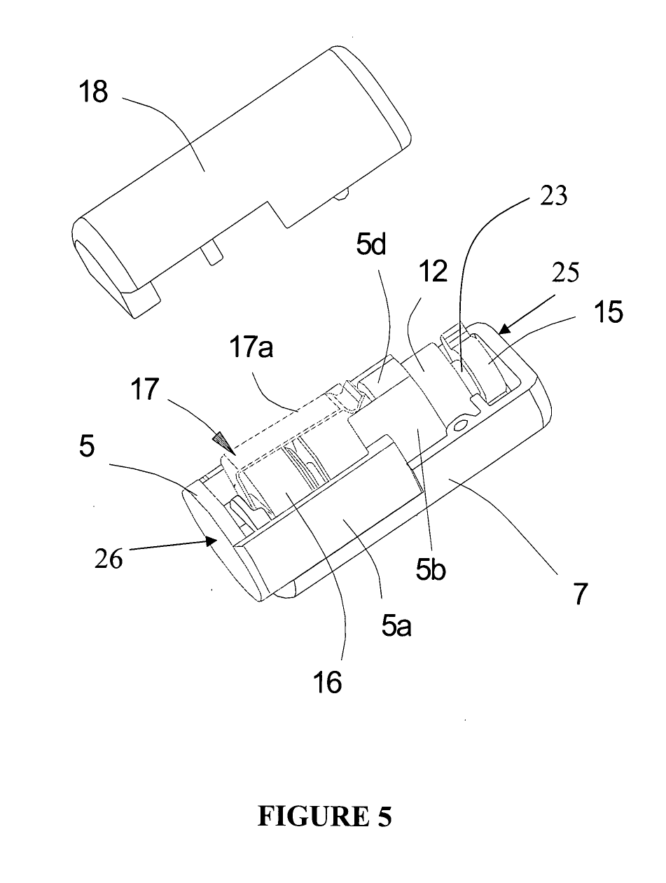

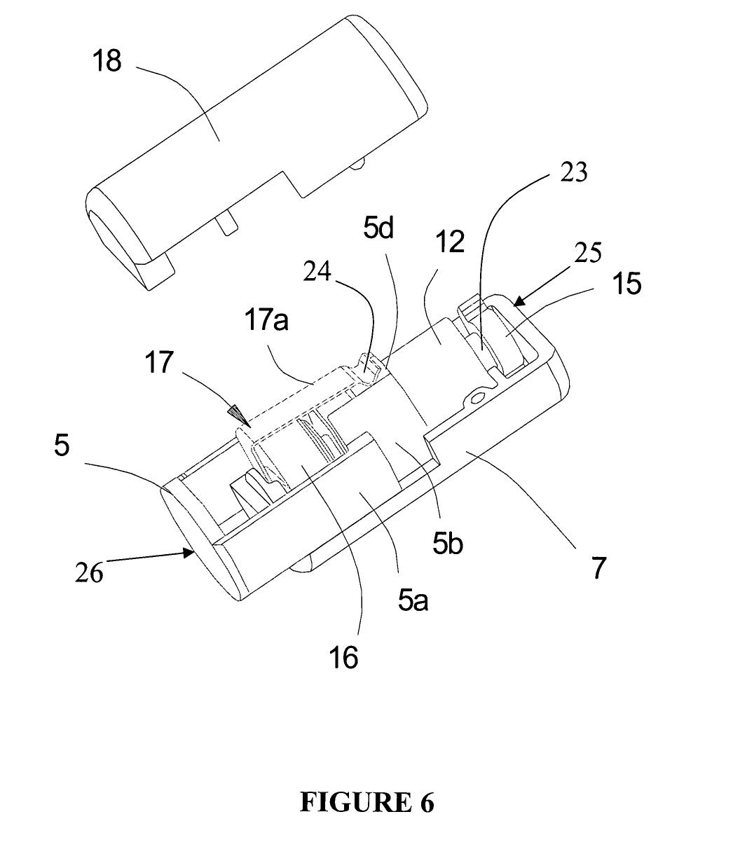

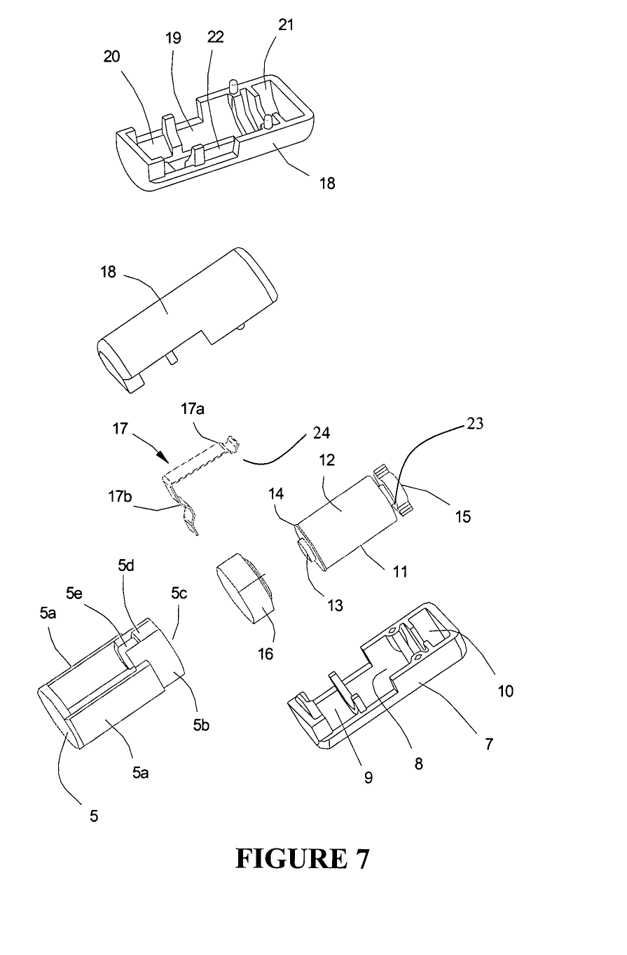

FIGS. 4, 5 and 6 illustrate perspective views of vibration generator 4 in accordance with an embodiment of the presently described invention. FIG. 7 illustrates an exploded view of vibration generator 4 in accordance with an embodiment of the presently described invention. In an embodiment, power switch 5 includes a plurality of arms 5a, an annular ring 5b and a recess 5d. Each of plurality of arms 5a is connected to an annular ring 5b. Thus, when arms 5a are moved upwards or downwards, ring 5b also moves upwards or downwards. Annular ring 5b is shaped so as to define a hole 5c. Annular ring 5b also includes recess 5d. In an embodiment, recess 5d extends to a slot 5e on an outer surface of ring 5b.

Vibration generator 4 includes a motor 11, an electrically conductive strip 17, a lower casing 7, an upper casing 18, a battery 16, an eccentric mass 15 and a plurality of cavities or hollow portions, including cavities 8, 9, 10, 19, 20 and 21.

Upper casing 18 includes a recess 22. Conductive strip 17 includes a switching arm 17a and a battery contact arm 17b. Lower casing 7 includes a cavity or hollow portion 8.

Motor 11 includes an outer surface 12 (which can act as a first electric pole 12 of motor 11), a second electric pole 13, a plate 14 and a shaft 23. Eccentric mass 15 and shaft 23 are configured so that mass 15 can be attached, connected or fixed to shaft 23.

Lower casing 7 comprises cavity 8 for accommodating motor 11. Lower casing 7 also comprises cavity 9 for accommodating battery 16. Lower casing 7 includes cavity 10 for providing a clearance to eccentric mass 15 on shaft 23 of motor 11.

Upper casing 18 comprises cavity 19 for accommodating motor 11, a cavity 20 for accommodating battery 16, cavity 21 for providing a clearance to eccentric mass 15 on shaft 23 of motor 11. Recess 22 in upper casing 18 is provided to locate arm 17a.

In an embodiment, hole 5c is snug fit to outer surface 12 of motor 11. That is, hole 5c is large enough to encircle outer surface 12 of motor 11 with little or no room or clearance for any other object between annular ring 5b and outer surface 12 of motor 11. However, in an embodiment, hole 5c is large enough to permit annular ring 5b to slide relative to and along a longitudinal axis of motor 11.

In an embodiment, outer surface 12 of motor 11 is made of an electrically conductive material. For example, outer surface 12 of motor 11 can comprise a metal, or metallic or other conductive material. In such an embodiment, outer surface 12 of motor can act as first electric pole 12 of motor 11. Second electric pole 13 of motor 11 is located on plate 14. In an embodiment, plate 14 is made of a non-conducting material. For example, plate 14 can be formed of a non-metallic or a plastic material. By “plastic” in the present description is meant a suitable synthetic or other hardened resin material or polymer of appropriate resilience and strength and other characteristics suited for the present purposes. Plate 14 can be connected or affixed to one end of motor 11.

In operation, when power switch 5 is pulled away from vibration generator 4 (as shown in FIG. 1), arms 5a are also pulled away from an end of motor 11 that is connected to shaft 23. As arms 5a are pulled away from this end of motor 11, ring 5b is pulled towards arm 17a of conductive strip 17. When ring 5b is pulled a sufficient distance towards strip 17, an end 24 of arm 17a closest to the end of motor 11 (that is, the end that is connected to shaft 23) is pulled up onto recess 5d of ring 5b. When arm 17a is pulled up onto recess 5d of ring 5b, a portion or all of recess 5d in annular ring 5b separates the arm 17a and outer surface 12 of motor 11 (which, as described above, also acts as first electric pole 12 of motor 11). That is, recess 5d separates conductive strip 17 (including arm 17a and end 24 of arm 17a) from contacting motor 12.

When arm 17a and outer surface 12 of motor 11 are separated, no voltage or power is supplied to motor 11 from battery 16. When no voltage or power is supplied to motor 11 from battery 16, generator 4 is in the OFF position and does not vibrate.

If power switch 5 is pushed towards vibration generator 4, the portion of recess 5d of annular ring 5b can be moved away from arm 17a and end 24 of arm 17a. This movement can cause arm 17a and/or end 24 of arm 17a to contact outer surface 12 of motor 11 (which, as described above, also can act as first electric pole 12 of motor 11). When switch 5 is pushed towards generator 4 and arm 17a contacts first electric pole 12 of motor, power or voltage is supplied to motor 11 from battery 16. When power or voltage is supplied to motor 11 from battery 16, generator 4 is in the ON position, becomes activated and begins vibrating.

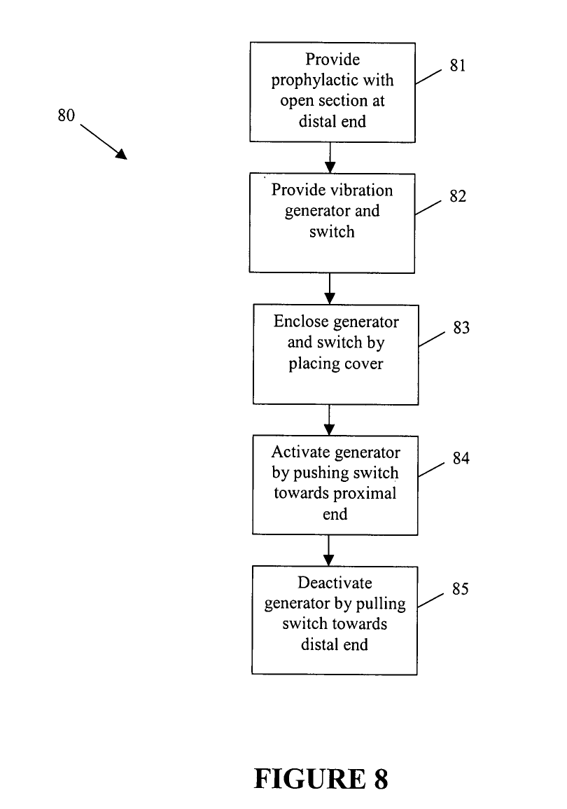

FIG. 8 illustrates a flowchart for a method 80 of providing a vibrating prophylactic in accordance with an embodiment of the presently described invention. In an embodiment, method 80 proceeds as follows. First, at step 81, a prophylactic is provided with an open section at its distal end, as described above. Next, at step 82, a vibration generator and activation or power switch for the generator are provided, also as described above. For example, generator 4 and switch 5 can be provided, as described above.

Next, at step 83, the generator and switch are enclosed in the prophylactic by inserting them into the open section at the distal end of the prophylactic and closing them in this end by placing a cover over them, as described above. For example, generator 4 and switch 5 can be placed in section 3 and enclosed using cover 6, as described above. In an embodiment, the generator and switch are provided at step 83 in the OFF, or deactivated, position. In another embodiment, the generator and switch are provided at step 83 in the ON, or activated, position

Next, at step 84, the generator is caused to vibrate by activating it. The generator can be activated by pushing the switch towards the proximal end 25 of the prophylactic, as described above. For example, generator 4 can be activated by pushing switch 5 towards the proximal end 25 of device 1.

In an embodiment, if the generator is provided at step 83 in the activated position, method 80 skips step 84. That is, in such an embodiment, method 80 proceeds from step 83 to step 85.

Next, at step 85, the generator is stopped from vibrating by deactivating it. The generator can be deactivated by pulling the switch towards the distal end of the prophylactic, as described above. For example, generator 4 can be deactivated by pulling switch 5 towards the distal end of device 1.

While particular elements, embodiments and applications of the presently described invention have been shown and described, it is understood that the presently described invention is not limited thereto since modifications may be made by those skilled in the technology, particularly in light of the foregoing teaching. It is therefore contemplated by the appended claims to cover such modifications and incorporate those features that come within the spirit and scope of the presently described invention.

What is claimed is:

1. A prophylactic device including a vibration generator and a switch disposed in a distal end of said device, said vibration generator activated by pushing said switch towards a proximal end of said device.

2. The device of claim 1, wherein said distal end of said device is curved along a direction transverse to a longitudinal axis of said device.

3. The device of claim 1, wherein said vibration generator is deactivated by pulling said switch towards said distal end of said device.

4. The device of claim 1, further including a battery, a conductive strip and a motor, said strip configured to connect said battery with said motor when said generator is activated.

5. The device of claim 4, wherein said switch includes a plurality of arms connected to a ring that surrounds at least a portion of said motor.

6. The device of claim 5, wherein said ring includes a recess configured to separate said strip from said motor when said switch is pulled towards said distal end of said device.

7. The device of claim 5, wherein said ring is configured to permit contact between said strip and said motor when said switch is pushed towards said proximal end of said device.

8. The device of claim 4, wherein said motor includes first and second electric poles, said first electric pole disposed at least a portion of a surface of said motor and said second electric pole disposed at a distal end of said motor.

9. The device of claim 1, further including a cover configured to be disposed in a groove of said device, said cover enclosing said generator and said switch in said distal end when disposed in said groove.

10. A switching apparatus for activating a vibration generator, said apparatus including:
a ring including a recess;
a switch including at least one arm connected to said ring; and
an electrically conductive arm configured to contact a pole of a motor when said ring is in an activated position and configured to contact said recess when said ring is in a deactivated position.

11. The apparatus of claim 10, wherein said switch is capable of moving relative to said arm to move said ring from said deactivated position to said activated position.

12. A method for providing a vibrating prophylactic device including:
providing a vibration generator and a switch in a distal end of said device; and
activating said vibration generator by pushing said switch towards a proximal end of said device.

13. The method of claim 12, wherein said providing step includes providing a curvature to said distal end of said device, wherein said distal end is curved along a direction transverse to a longitudinal axis of said device.

14. The method of claim 12, further including deactivating said vibration generator by pulling said switch towards said distal end of said device.

15. The method of claim 12, further including providing a battery, a conductive strip and a motor, said strip configured to connect said battery with said motor when said activating step occurs.

16. The method of claim 15, wherein said step of providing said vibration generator and said switch includes providing said switch with a plurality of arms connected to a ring that surrounds at least a portion of said motor.

17. The method of claim 16, further including deactivating said vibration generator by pulling said switch towards said distal end of said device and causing a recess in said ring to separate said strip from said motor.

18. The method of claim 16, wherein said activating step includes contacting said strip and said motor by moving at least one of said ring and a recess in said ring from an end of said strip.

19. The method of claim 15, wherein said step of providing said motor includes providing said motor with first and second electric poles by disposing said first electric pole at least a portion of a surface of said motor and disposing said second electric pole at a distal end of said motor.

20. The method of claim 12, further including enclosing said generator and said switch in said distal end of said device with a cover configured to be disposed in a groove of said device.
